SEQUENCE LISTING (8 sequences)

SEQ ID NO: 1 - DNA, 30 bp, Homo sapiens
  LN1PF
ccttccacttcccccgggagctcagccgcg

SEQ ID NO: 2 - DNA, 30 bp, Homo sapiens
  LN1PR
cgcggctgagctcccgggggaagtggaagg

SEQ ID NO: 3 - DNA, 24 bp, Mus musculus
  mHes1 forward
tcctaacgcagtgtcaccttccag

SEQ ID NO: 4 - DNA, 24 bp, Mus musculus
  mHes1 reverse
ccaagttcgtttttagtgtccgtc

SEQ ID NO: 5 - DNA, 24 bp, Mus musculus
  mHey1 forward
caggagggaaaggttattttgacg

SEQ ID NO: 6 - DNA, 26 bp, Mus musculus
  mHey1 reverse
tagttgttgagatgggagaccaggcg

SEQ ID NO: 7 - DNA, 21 bp, Mus musculus
  18S forward
agtccctgccctttgtacaca

SEQ ID NO: 8 - DNA, 21 bp, Mus musculus
  18S reverse
gatccgagggcctcactaaac

(19) United States
(12) Patent Application Publication
(10) Pub. No.: US 2021/0196681 A1
(43) Pub. Date: Jul. 1, 2021

(54) SELECTIVE PRESENILIN-2 GAMMA-SECRETASE INHIBITORS

(71) Applicants: UNIVERSITEIT MAASTRICHT, MAASTRICHT (NL); ACADEMISCH ZIEKENHUIS MAASTRICHT, MAASTRICHT (NL); AUCKLAND UNISERVICES LIMITED, AUCKLAND (NZ)

(72) Inventors: Marc Antoine Gijbert Gilles Vooijs, Cadier En Keer (NL); Adrianus Johannes Groot, Maastricht (NL); Jeffrey Bruce Smaill, Auckland (NZ); Patrick David O'Connor, Auckland (NZ); Amir Ashoorzadeh, Auckland (NZ)

(21) Appl. No.: 17/271,858

(22) PCT Filed: Aug. 27, 2019
(86) PCT No.: PCT/EP2019/072876

§ 371 Date: Feb. 26, 2021

(30) Foreign Application Priority Data
Aug. 27, 2018 (EP) 18190985.4

(51) Int. Cl. A61K 31/417; A61P 35/00; A61K 31/4706

(52) U.S. Cl. CPC A61K 31/417; A61K 31/4706; A61P 35/00

(57) ABSTRACT

The present invention relates to selective presenilin-2 γ-secretase inhibitors for use in the treatment of various diseases associated with a defect leading to Notch receptor hyperactivity. In particular the present invention relates to selective presenilin-2 γ-secretase inhibitors for use as a highly selective anti-cancer treatment. Preferred selective presenilin-2 γ-secretase inhibitors include small molecules, like 4-aminoquinolines or imidazole compounds, (monoclonal) antibodies and known γ-secretase inhibitors or analogues and combinations thereof.

### CROSS-REFERENCE TO RELATED APPLICATIONS

This application is a national phase entry under 35 U.S.C. § 371 of International Patent Application PCT/EP2019/072876, filed Aug. 27, 2019, designating the United States of America and published in English as International Patent Publication WO 2020/043736 A1 on Mar. 5, 2020, which claims the benefit under Article 8 of the Patent Cooperation Treaty to European Patent Application Serial No. 18190985.4, filed Aug. 27, 2018, the entireties of which are hereby incorporated by reference. The Notch signalling pathway is an evolutionarily conserved cascade that regulates the development and homeostasis of a variety of tissues. Consistent with its central role in regulating these events, aberrant Notch signalling is often causative for a range of diseases including cancer. The cellular processes that it controls include, but are not restricted to, the maintenance and differentiation of stem cells, cell fate determination, cell-cycle regulation, cell death, and angiogenesis. Thus Notch signalling affect many important hallmarks of various diseases, including cancer.

Notch signal transduction is initiated by ligation of Notch receptors with ligands expressed on neighbouring cells. Mammals possess four Notch receptors, i.e. Notch1, Notch2, Notch3 and Notch4. Mammals further possess five ligands, i.e. Jagged1 (Jag1), Jagged2 (Jag2), Delta-like ligand 1 (Dll1), Delta-like ligand 3 (Dll3) and Delta-like ligand 4 (Dll4). The receptors are synthesized as single precursor proteins that are cleaved during transport to the cell surface, where they are expressed as heterodimers. Ligand-receptor interaction between two neighbouring cells results in two successive proteolytic cleavages. This process of sequential cleavage of membrane signalling molecules is termed regulated intramembrane proteolysis (RIP).

The first step of the sequential cleavage of membrane signalling molecules, Notch precursors are cleaved, during maturation in the trans-Golgi network, by a furin-like convertase producing a heterodimeric receptor with the Notch extracellular domain (NECD) non-covalently bound to a transmembrane/intracellular fragment (TMIC).

The second proteolytic cleavage is mediated by a metalloprotease of the ADAM family (ADAM10), which cleaves the receptor in the extracellular domain, close to the transmembrane domain. The released extracellular domain is then transendocytosed by the ligand-expressing cell.

The third cleavage occurs within the transmembrane (TM) domain and is mediated by the γ-secretase activity of a multiprotein complex comprised of four major subunits, i.e. presenilin (Psen), nicastrin (NCT), anterior pharynx-defective 1 (Aph1), and presenilin enhancer 2 (PEN-2). Biochemical analysis has shown that the four major subunits, i.e. NCT, PEN-2, Psen and Aph1 are required and sufficient for robust γ-secretase activity. The final cleavage mediated by the γ-secretase complex liberates the cytoplasmic domain of the Notch receptor (NICD), which subsequently translocates to the nucleus, where it binds to its downstream transcription factor RBP-Jk/CSL and thereby activates transcription (see: FIG. 5).

The γ-secretase complex is known for processing of amyloid precursor protein (APP), causal to Alzheimer's disease. As already described above, the γ-secretase complex is further known for processing of the Notch family proteins frequently deregulated and mutated in cancers and other disorders. However, clinical use of γ-secretase inhibitors for Alzheimer's disease as well as in cancer treatment has been hampered due to adverse side effects, caused by the inhibition of the physiological (wild-type) Notch signalling pathway.

The proteins in the γ-secretase complex are heavily modified by proteolysis during assembly and maturation of the complex; a required activation step is in the autocatalytic cleavage of Psen to N- and C-terminal fragments. Psen, an aspartyl protease, is the catalytic subunit and its catalytic activity is required for Notch and APP cleavage. Mutations in the Psen gene have been shown to be a major genetic risk factor for Alzheimer's disease. NCT's primary role is in maintaining the stability of the assembled complex and regulating intracellular protein trafficking. PEN-2 associates with the complex via binding of a transmembrane domain of Psen and, among other possible roles, helps to stabilize the complex after Psen endo-proteolysis has generated the activated N-terminal and C-terminal fragments. Aph1, which is required for proteolytic activity, binds to the complex via a conserved alpha helix interaction motif and aids in initiating assembly of premature components.

In humans, two homologs of both Psen and Aph1 have been identified in the genome, i.e. Psen homolog 1 (Psen1), Psen homolog 2 (Psen2), Aph1 homolog A (Aph1A) and Aph1 homolog B (Aph1B). The contribution of each of the different possible γ-secretase complexes to substrate (Notch) specificity and enzyme activity is unknown.

There are more than 150 mutations known in Psen1 and Psen2, which alter the processing of APP, associated with early-onset familial Alzheimer's disease. As such, γ-secretase inhibitors are widely explored as therapeutics for targeting Alzheimer's disease. Also, γ-secretase inhibitors are investigated as a strategy to block oncogenic Notch signalling in T cell acute lymphocytic leukemia's and many solid cancers. Unfortunately, the clinical implementation of γ-secretase inhibitors has been hampered by dose-limiting toxicities caused in normal tissues, most notably the skin, thymus, gut and brain in mice and humans. The intestinal toxicities are largely related to loss of function of Notch1 and Notch2 receptor signalling.

The present invention now provides the insight that γ-secretase inhibitors selectively inhibiting Psen2 γ-secretase (selective Psen2 γ-secretase inhibitors) can be selected, in order to provide a highly selective treatment of various diseases associated with mutations, alterations, or wild-type deviations affecting Notch signalling pathways, i.e. a disease associated with an aberrant Notch signalling pathway. More in particular, the present invention provides selective Psen2 γ-secretase inhibitors for use in the treatment of a disease associated with a defect leading to Notch receptor hyperactivity.

As used herein, the term “aberrant Notch signalling pathway” may refer to gain-of-function mutations affecting the activity of the Notch receptor. Such gain-of-function mutations may relate to gain-of-function mutations of the Notch receptor itself or may also refer to loss-of-function or gain-of-function mutations (non-Notch receptor mutations) affecting the activity of the Notch receptor (mutations resulting in Notch receptor hyperactivity). Even further, the term “aberrant Notch signalling pathway” as defined herein may also relate to a defect leading to Notch receptor hyperactivity caused by chromosomal rearrangements in the Notch signalling pathway or by defects in protein trafficking altering the subcellular localisation of the Notch receptor molecules.

In an embodiment of the present invention, the defect leading to Notch receptor hyperactivity may be caused by oncogenic, e.g. oncogenic gain-of function, mutations of the Notch receptor itself or may be caused by other factors, e.g. gain-of-function or loss-of-function mutations other than mutations of the Notch receptor itself, affecting the Notch signalling pathway, i.e. the activity of the Notch receptor.

The disease associated with an aberrant Notch signalling pathway may be selected from the group consisting of a disease associated with an aberrant Notch1 signalling pathway, a disease associated with an aberrant Notch2 signalling pathway, a disease associated with an aberrant Notch3 signalling pathway and a disease associated with an aberrant Notch4 signalling pathway. In an embodiment of the present invention the disease associated with an aberrant Notch signalling pathway is a disease associated with a defect leading to Notch1, Notch2, Notch3 and/or Notch4 receptor hyperactivity. In a further embodiment, the disease associated with an aberrant Notch signalling pathway is cancer.

It was found that by using loss-of-function and gain-of-function approaches either one of Aph1A and Aph1B genes is necessary for Notch proteolysis and that, however, different Psen γ-secretase complexes produce characteristic Notch cleavage profiles and distinct effects on target gene activation. The present invention is supported by the finding that Psen2:Aph1A or Psen2:Aph1B γ-secretase complexes play a major role in processing ligand-independent mutated Notch receptors, e.g. oncogenic ligand-independent Notch1 receptor. At the same time, Psen2:Aph1A or Psen2:Aph1B γ-secretase complexes play a minor role to cleave physiological Notch receptors. It was further found that the Psen2:Aph1B γ-secretase complex, in particular, efficiently process oncogenic ligand-independent Notch1 receptors, but is attenuated in its ability to cleave wild type Notch1 receptors.

The present invention relates to selective Psen2 γ-secretase inhibitors for use in the treatment of diseases associated with a defect leading to Notch receptor hyperactivity, e.g. mutations in Notch signalling pathways, including cancers, skin diseases, allergic disorders, neurological disorders, immune disorders, cognitive symptoms, congenital disorders, skeletal disorders, haematological malignancies, and respiratory diseases. It is noted that diseases associated with altered Notch signalling pathways, i.e. having an increased Notch receptor activity, may also include diseases associated with chromosomal rearrangements in Notch signalling pathways or diseases associated with defects in protein trafficking altering the subcellular localisation of Notch receptor molecules. These genes include, but not restricted to, NUMB, Lethal giant discs (Lgd), endosomal sorting complexes required for transport (ESCRT) family proteins and several E3-ubiquitin ligases including, but not restricted to, Deltex and suppressor of Deltex, Scribble complex genes including, but not restricted to, Lethal giant Larvae (lgl) or alterations/modifications in the Notch signalling pathway that promote intra-endosomal Delta/Notch signalling.

Cancers associated with a defect leading to Notch receptor hyperactivity, e.g. mutations in Notch signalling pathways or trafficking pathways affecting Notch localisation, may include leukemia, breast cancer, prostate cancer, colorectal cancer, penile cancer, Head and Neck Cancer, lung cancer, oesophageal cancer, liver cancer, ovarian, cervical and endometrial cancer, brain cancer, bladder cancer, skin cancer, bone cancer, disseminated diseases, such as metastatic cancer, and the like.

For example, Notch1 hyperactivity associated cancers may include basal type breast cancer (e.g. mammary tumours), bone cancer (e.g. human osteosarcoma), and skin cancer (e.g. nonmelanoma skin cancer including most frequent basal cell carcinoma (BCC), and second most common squamous cell carcinoma (SCC)).

Allergic disorders associated with a defect leading to Notch receptor hyperactivity, e.g. mutations in Notch signalling pathways or trafficking pathways affecting Notch localisation, may include asthma.

Neurological disorders associated with a defect leading to Notch receptor hyperactivity, e.g. mutations in Notch signalling pathways or trafficking pathways affecting Notch localisation, may include central nervous system malignancies.

Congenital disorders associated with a defect leading to Notch receptor hyperactivity, e.g. mutations in Notch signalling pathways or trafficking pathways affecting Notch localisation, may include Notch2 associated Hajdu-Cheney syndrome and serpentine fibula polycystic kidney syndrome, and Notch3 associated infantile myofibromatosis.

Skeletal disorders associated with a defect leading to Notch receptor hyperactivity, e.g. mutations in Notch signalling pathways or trafficking pathways affecting Notch localisation, may include Notch3 associated lateral meningocele syndrome (Lehman syndrome), breast cancer stem cells skeletal invasiveness, and osteolytic potential.

Haematological malignancies associated with a defect leading to Notch receptor hyperactivity, e.g. activating mutations in Notch signalling pathways or trafficking pathways affecting Notch localisation, may include Notch1 associated T-cell acute lymphoblastic leukaemia (T-ALL), and B-cell chronic lymphocytic leukaemia, and Notch2 associated B-cell lymphoma among others.

Respiratory diseases associated with a defect leading to Notch receptor hyperactivity, e.g. mutations in Notch signalling pathways or trafficking pathways affecting Notch localisation, may include Notch associated asthma, pulmonary fibrosis, and non-small cell lung carcinoma.

Selective Psen2 γ-secretase inhibitors are preferably selective in inhibiting the Psen2:Aph1B γ-secretase complex. It was found that by inhibiting the Psen2:Aph1B γ-secretase complex selectively, oncogenic ligand-independent Notch receptors, in particular oncogenic ligand-independent Notch1 receptors, can be efficiently inhibited without significantly affecting physiological Notch receptors, such as physiological Notch1 receptors (and other physiological Notch family receptors).

Further, selective Psen2 γ-secretase inhibitors may comprise small molecule inhibitors, including γ-secretase inhibitors (GSIs), small interfering RNA (siRNA), or other approaches inhibiting Psen2 mRNA transcription and translation and (monoclonal) antibodies (mAb). Optionally, the selective Psen2 γ-secretase inhibitors of the present invention may be administered in combination with other cytostatic agents including small molecules and antibodies and ionizing radiation (Radiotherapy).

The present invention further relates to a selective Psen2 γ-secretase inhibitor for use in the treatment of a disease associated with a defect leading to Notch receptor hyperactivity, e.g. an aberrant Notch signalling pathway, wherein the Psen2 γ-secretase inhibitor is identified by a screening method comprising the steps of:
providing a candidate for selectively inhibiting Psen2 γ-secretase complex;
contacting the candidate with a Psen1 γ-secretase complex and Psen2 γ-secretase complex; and
identifying the candidate selectively inhibiting Psen2 γ-secretase as the selective Psen2 γ-secretase inhibitor.

The present invention further relates to 4-aminoquinolines as selective Psen2 γ-secretase inhibitors for use in the treatment of a disease associated with a defect leading to Notch receptor hyperactivity, i.e. mutations affecting the Notch signalling pathway, wherein the 4-aminoquinolines are selected from compounds of formula:

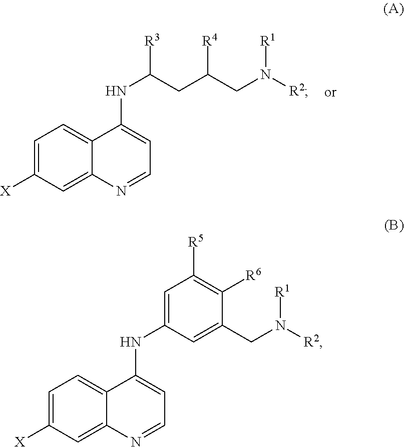

wherein:

X is selected from hydrogen, fluoro, chloro, bromo, iodo, hydroxy, nitro, cyano, amino, C₁₋₄ alkyl, C₁₋₄ alkoxy, C₁₋₄ alkylamino, C₁₋₄ alkylthio, aryl, benzyl, phenoxy, or benzoxy;

R¹ and R² are independently selected from hydrogen, C₁₋₄ alkyl, aryl, benzyl, C₁₋₄ alkylol, or alkylphenol;

R³ and R⁴ are independently selected from hydrogen, C₁₋₄ alkyl, aryl, benzyl, or C₁₋₄ alkylol; and

R⁵ and R⁶ are independently selected from hydrogen, fluoro, chloro, bromo, iodo, hydroxy, nitro, cyano, amino, or C₁₋₄ alkyl.

In an embodiment of the present invention the 4-aminoquinolines as selective Psen2 γ-secretase inhibitors may be selected from compounds of formula A or B, wherein:

X is selected from chloro;

R¹ and R² are independently selected from ethyl or 2-hydroxyethyl;

R³ and R⁴ are independently selected from hydrogen or methyl; and

R⁵ and R⁶ are independently selected from hydrogen or hydroxyl.

Preferred 4-aminoquinolines as selective Psen2 γ-secretase inhibitors are selected from the group consisting of amodiaquine, chloroquine, and hydroxychloroquine.

It was found that by providing a 4-aminoquinoline, such as chloroquine, in further combination with a γ-secretase inhibitor, e.g. a non-selective Psen2 γ-secretase inhibitor or another selective Psen2 γ-secretase inhibitor, the amount of 4-aminoquinoline, such as chloroquine, can be significantly reduced without affecting the therapeutic activity of the 4-aminoquinoline, such as chloroquine, due to a synergistic effect provided by the combination of the 4-aminoquinoline and a γ-secretase inhibitor. The present invention thus provides a highly effective dosage form comprising the 4-aminoquinoline of the present invention, such as chloroquine, in combination with a γ-secretase inhibitor, e.g. a non-selective Psen2 γ-secretase inhibitor or a further selective Psen2 γ-secretase inhibitor. The dosage form of the present invention has reduced side-effects without affecting the therapeutic activity of the active compounds.

The present invention also relates to imidazole compounds as selective Psen2 γ-secretase inhibitors, wherein the imidazole compounds are selected from compounds of formula:

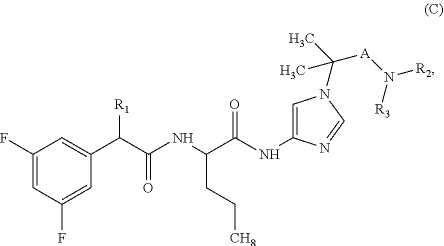

wherein:

R₁ is selected from hydrogen, or hydroxy;

A is selected from —CH₂— or —CO—; and

R₂ and R₃ are independently selected from hydrogen, methyl, ethyl, propyl, isopropyl, tert-butyl, or neopentyl,

wherein in case R₁ is hydrogen, A is —CO— and R₂ is hydrogen,
with the proviso that when R₁ is hydrogen, A is —CO—.

In an embodiment of the present invention the imidazole compounds as selective Psen2 γ-secretase inhibitors may be selected from compounds of formula C, wherein:

R₁ and R₂ are selected from hydrogen, A is selected from —CO— and R₃ is selected from neopentyl; or

R₁ is selected from hydroxyl, A is selected from —CH₂—, and R₂ and R₃ are selected from methyl.

In a further embodiment of the present invention the imidazole compounds as selective Psen2 γ-secretase inhibitors may be selected from the group consisting of:
(S)-2-((S)-2-(3,5-difluorophenyl)-2-hydroxyacetamido)-N-(1-(1-(dimethylamino)-2-methylpropan-2-yl)-1H-imidazol-4-yl)pentanamide,
(S)-2-((R)-2-(3,5-difluorophenyl)-2-hydroxyacetamido)-N-(1-(1-(dimethylamino)-2-methylpropan-2-yl)-1H-imidazol-4-yl)pentanamide, and
(S)-2-(2-(3,5-difluorophenyl)-acetamido)-N-(1-(2-(methyl-1-(neopentylamino)-1-oxopropan-2-yl)-1H-imidazol-4-yl)pentanamide.

The present invention further relates to the above imidazole compounds for use as a medicament. In a further embodiment, the present invention relates to the above imidazole compounds for use in the treatment of a disease associated with a defect leading to Notch receptor hyperactivity, such as cancer. Even further, the present invention relates to the above imidazole compounds for use in the treatment of a disease associated with a defect leading to Notch1 receptor hyperactivity. In a further embodiment, the present invention relates to a pharmaceutical composition comprising an imidazole compound according to the present invention.

### EXPERIMENTAL PROCEDURES

Cell Lines and Chemicals

Psen1, Psen2 double knockout/Aph1a, Aph1b, Aph1c triple Knockout (QKO) mouse embryonic fibroblasts (MEFs) were generated and maintained in DMEM/F12+10% FCS+0.1% PEN/STREP. Cells were treated for 24 hours with dibenzazepine (DBZ) 0.2 μM (Syncom, Groningen, The Netherlands) and DMSO unless stated otherwise. 1 μg/ml of rDll4-Cf (R&D Systems) or rDll1 (kind gift from I. Bernstein), 0.2% gelatine 0.1% BSA in PBS was coated overnight at 4° C., rinsed once with PBS before cells were plated.

Plasmids, Transfections and Infections

Murine ΔEGF-Notch1-L1594P-6xMYC (LNR) was subcloned into the pBabe-Puro vector and was transfected together with pCMV-VSVG and pVPack-GP into HEK293FT cells to produce pseudo-lenti viruses to infect: QKO, Psen1:Aph1A γ-secretase complex (1A), Psen1:Aph1B γ-secretase complex (1B), Psen2:Aph1A γ-secretase complex (2A) and Psen2:Aph1B γ-secretase complex (2B) cells. Cells transduced with ΔEGF-Notch1-L1594P construct were maintained with 1 μg/ml of puromycin. pcDNA3 hNotch1 full-length construct, encoding an N terminal FLAG-tag, followed by the ectodomains and the transmembrane domains of Notch1 fused to the Gal4 DNA binding domain and the transcriptional activation domain of Notch1 was used. This plasmid was used as a template to generate the L1594P mutant construct with primers forward LN1PF: 5′-CCTTCCACTTCCCCCGGGAGCTCAGCCGCG-3′ and reverse LN1PR: 5′-CGCGGCTGAGCTCCCGGGGGAAGTGGAAGG-3′ by QuickChange site directed mutagenesis according to manufacturer's instructions (Stratagene) and was sequence verified.

Firefly luciferase encoding reporter plasmids containing synthetic CSL binding sites (pGL4.24-12xCSL) was used to monitor Notch activity, using pGL4.74 TK-hRL (Promega) as transfection control. 12XCSL Gaussia was generated by subcloning a fragment of synthetic 12xCSL with minimal CMV promoter into pGLuc-Basic Vector from New England Biolabs. A CMV driven Firefly luciferase-GFP construct was used as transfection control. Transfections were performed using Fugene HD transfection reagent (Promega).

Ligand Stimulation Assay

Cells were plated on either rDll4 or rDll1 coated plates and treated with either DMSO or DBZ for 24 hours. Co-culture with ligand was used to activate endogenous Notch1 in MEFs.

Reporter Assays Cells were plated 24 hours before transfecting with two reporter genes.

1 μg of the primary reporter was used to record the effect of specific stimuli and 0.1 μg of the control reporter for normalization. 24 hours post-transfection cells were replated into 24 well plates and treated as indicated for 24 hours. Cells were washed, lysed and luciferase was measured, as described by manufacturer (dual-glo luciferase reporter assay system Promega) on a Fluostar Omega plate reader (BMG Labtech) For Gal4-luciferase assay, various concentrations of the Notch receptor plasmids were transfected and complemented with empty vector. A plasmid containing synthetic Gal4 binding sites (5xUAS FR-luc) was used together with a thymidine kinase driven Renilla luciferase plasmid to normalize the data. For Gaussia-Luc assay, medium was collected at different time points to monitor overtime 12xCSL activation and measured with the BioLux® Gaussia Luciferase Assay Kit according to manufacturer's instructions (NEB). All values are expressed as relative light units (RLU).

Western Blotting and Antibodies

Cells were directly lysed in Laemmli buffer and samples were boiled prior to resolving by SDS-PAGE. Proteins were transferred onto nitrocellulose membranes and blocked for 1 hour in 5% skim milk in TBS, 0.05% Tween-20 (TBS-T). Membranes were probed overnight at 4° C. with primary antibodies and bound antibodies were visualized using HRP-linked secondary antibodies (Cell-Signalling) and ECL Luminescence (Pierce Biotechnology). Anti-Lamin A/C (1:1000) and anti-beta-tubulin III (1:1500) were purchased from Sigma-Aldrich, anti-Notch1 S3-Val1744 (1:1000) and secondary anti-mouse and anti-rabbit (1:2500) were from Cell Signaling. Anti-Myc 9E10 (1:5000) and anti-Notch1 C20 (1:1000) were from Santa Cruz Biotechnology.

qRT-PCR-RNA

Isolation was performed according to manufacturer's instructions (Nucleospin RNA isolation, Macherey Nagel) and cDNA synthesis was performed using I-script reverse transcriptase (Bio-RAD). mRNA expression was normalized to 18S. Primers used are described in Table 1.

TABLE 1
Used qRT-PCR primers

| | |
|---|---|
| mHes1 | 5′ TCCTAACGCAGTGTCACCTTCCAG 3′ |
| forward | |
| mHes1 | 5′ CCAAGTTCGTTTTTAGTGTCCGTC 3′ |
| reverse | |
| mHey1 | 5′ CAGGAGGGAAAGGTTATTTTGACG 3′ |
| forward | |
| mHey1 | 5′ TAGTTGTTGAGATGGGAGACCAGGCG 3′ |
| reverse | |
| 18S forward | 5′ AGTCCCTGCCCTTTGTACACA 3′ |
| 18S reverse | 5′ GATCCGAGGGCCTCACTAAAC 3′ |

Statistical Analysis

All error bars represent mean±SEM of three independent experiments. The Student's t-test was used for the analysis.

Results

Psen and Aph1 Subunits Produce Different Notch1 Cleavage Profiles and Activity

To directly investigate the contribution of different γ-secretase complex subunits on Notch signalling activity quintuple deficient (Aph1A−/−, Aph1B−/−, Aph1C−/−, Psen1−/−, Psen2−/− (QKO)) mouse embryonic fibroblasts (MEFs) reconstituted with 4 different combinations of human Aph1 cDNA (Aph1A or Aph1B) and Psen homologues (Psen1 or Psen2) were compared. QKO and reconstituted cells were grown on plates with bound recombinant Dll4, a potent inducer of Notch1 cleavage and transcriptional activity to monitor the effects on Notch1 cleavage. A C-terminal antibody (C-20) was used, as well as the Val1744 neo-epitope antibody, which detects γ-secretase cleaved Notch1 at valine1744.

Reconstitution with both Psen and Aph1 resulted in Notch1 γ-secretase cleavage for all combinations in response to Dll4 stimulation, albeit to a different extent. Consistently in QKO, no Notch1 cleavage at Val1744 was observed. Marked differences were observed in the extent of Val1744 cleavage, where 1B and 2A cells show robust cleavage, 1A cells show less cleavage and Val1744 cleavage was strongly reduced in 2B cells (FIG. 1A).

The differences observed in Notch receptor processing by the different γ-secretase complexes did not depend on the type of ligand used for stimulation, as similar results were obtained with recombinant Dll1 stimulation with Val1744 cleavage (FIG. 1B).

The C-20 antibody is not sensitive enough to detect NICD fragments and therefore only indirectly estimate S3 cleavage by assessing S2 accumulation in the presence of the γ-secretase inhibitor DBZ. Only minor differences in S2 accumulation were observed, which did not correlate with the strong differences observed in Val1744 cleavage. These findings were replicated in an independently generated series of MEFs reconstituted with 1A, 1B, 2A and 2B (FIG. 1).

Total Notch1 Processing by γ-Secretase does not Correlate with Cleavage at Valine 1744

To directly assess the effects of Aph1 and Psen Notch1 γ-secretase cleavage irrespective of cleavage at neo-epitope Val1744 we transfected MEFs with Notch1-Gal4 fusion proteins and co-cultured these with Dll4. Ligand induced γ-secretase of Notch1 will lead to the release of a Gal4-NICD fusion protein which can activate a reporter containing Gal4-DNA binding sites driving firefly luciferase expression. Thus, total γ-secretase cleavage of Notch1 (irrespective of cleavage site), leading to release of Gal4-NICD from the membrane is monitored in this assay. Upon Dll4 stimulation, transcriptional activity was induced in all the four cell lines reconstituted with different combinations of Psen and Aph1, in a dose dependent manner for Notch1 FL-Gal4 (FIG. 1C). In the absence of Psen and Aph1 (QKO), or ligand, no transcriptional activity was observed. Notably, the induction of Gal4 transcriptional activity was highly dependent on the composition of the γ-secretase complex.

Notch1 expressing cells showed highest Gal4 transcriptional reporter activity in Psen2:Aph1B (2B) MEFs, whereas transcriptional induction was lower across all doses in 1A, 1B and 2A MEFs, which showed comparable activity. This finding suggests that Notch1-Val1744 cleavage assessed by immunoblotting does not correlate to total S3 cleavage after ligand stimulation.

Different γ-secretase complexes produce different transcriptional output Dll4 induced a γ-secretase dependent increase in NOTCH/RBP-JK reporter luciferase activity in all cell lines, compared to the QKO cells upon Dll4 stimulation (FIG. 1D). Consistently, no ligand induced Notch transcriptional activity was observed in Y-secretase deficient cells (QKO). The lowest Notch dependent transcriptional activation was observed in cells reconstituted with Psen2, whereas activation in cells reconstituted with Psen1:Aph1B were several fold higher.

Dynamic time-course experiments using a Notch transcriptional reporter expressing secreted Gaussia-Luc reporter demonstrated a linear increase of Notch1 transcriptional activity in all combinations excluding time-dependent differences in transcriptional regulation missed in endpoint assays (FIG. 1E). A trend was found towards a higher Notch dependent transcriptional activation in cells reconstituted with Psen1:Aph1B.

To directly determine the effect of the different γ-secretase complexes on Dll4 induced target gene activation, we assessed mRNA expression of two well-known Notch target genes Hes1 and Hey1. We observed that Dll4 stimulation induced similar levels of Hes1 mRNA in all cell lines in comparison to QKO cells, independent of the γ-secretase subunit composition. In contrast, Hey1 mRNA expression was induced in 1A, 2A and 2B cells, but not in 1B cells after Dll4 stimulation (FIG. 1F). Thus, ligand induced activation of endogenous Notch1 target genes in cells does not correlate qualitatively nor quantitatively with S3/γ-secretase cleavage (Val1744) and differs dramatically with subunit composition.

Psen2:Aph1Bγ-Secretase Complexes Efficiently Process Oncogenic Notch Receptor

QKO, 1A, 1B, 2A and 2B MEFs expressing a constitutively active ligand-independent Notch1 mutant (ΔEGF-Notch1-L1594P-MYC) were generated. The oncogenic Notch1 T-ALL receptor was equally expressed in all cell lines as shown by anti-MYC immunoblotting, and processed by all four γ-secretase complexes, as shown by Val1744. NICD1 processing at Val744 cleavage was most strong in 1A and 2B cells and less pronounced in 1B and 2A cells, which inversely correlates with Val1744 cleavage in Notch1 wild type cells. DBZ blocked NICD1-Val1744 irrespective of the γ-secretase complex. In the absence of DBZ accumulation of S2 cleaved and full-length unprocessed Notch1 was observed indicative of blocked γ-secretase cleavage in 2A cells which further accumulated with DBZ (FIG. 2A).

To monitor all γ-secretase/S3 cleavage of oncogenic Notch1 receptors, MEFs were transfected with Notch1-L1594P-Gal4 and transcriptional activity was measured. Notch1-L1594P expression induced transcriptional activation in a dose dependent manner in 1A, 1B, 2A and 2B cells compared to QKO cells, where no induction was observed (FIG. 2B). In contrast to ligand induced cleavage of wildtype Notch1, total γ-secretase cleavage resembled Val1744 cleavage in a dose-dependent manner in cells expressing oncogenic Notch1. To answer whether differences in Val1744 cleavage of oncogenic Notch1 by different γ-secretase complexes also results in varying transcriptional outputs we performed 12xCSL reporter assays in MEFs expressing ΔEGF-Notch1-L1594P-MYC. Transcriptional activation of 12xCSL was induced in all cell lines expressing the mutant L1594P receptor, but not in QKO cells. Markedly, γ-secretase dependent Notch1 cleavage was up to an order of magnitude higher in QKO cells reconstituted with Psen2:Aph1B (FIG. 2C) compared to cells expressing other GS complexes.

The cleavage and transcriptional induction of mutant Notch1 proteins by different γ-secretase complexes in cells was stable over time and was also reflected in the activation of Hes1 and Hey1 mRNA target genes (FIG. 2D, 2E). Taken together these data demonstrate that different γ-secretase complexes that differently impact on physiological versus oncogenic Notch1 in cells.

### Example 1

Endosomal Trafficking Regulates the Processing of Oncogenic Notch1 Receptor by Psen2:Aph1B γ-Secretase Complexes

The endocytic pathway has emerged as an alternative way to regulate the transport and processing of Notch receptors. Chloroquine (CQ), a lysosomotropic agent that alters lysosomal pH and blocks vesicular fusion was used to investigate the contribution of this vesicular pathway to physiological and oncogenic Notch activity. Regardless of the γ-secretase complex in cells, it was observed that ligand induced wild type Notch1 signaling was not affected by the addition of CQ (FIG. 3A). In cells expressing mutant Notch1 it was observed that cells reconstituted with Psen2 were most strongly inhibited by CQ compared to Psen1 expressing cells. Moreover, mutant Notch1 activity in Psen2:Aph1B cells was up to 8-fold more sensitive to CQ inhibition than oncogenic Notch1 processed by Psen1 expressing cells.

### Example 2

Synthesis of Imidazole Compounds

The following imidazole compounds (table 2) were synthesized using the following general reaction scheme:

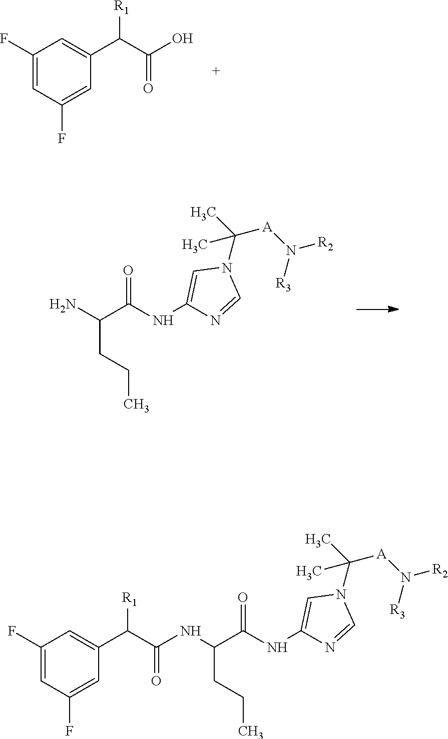

TABLE 2
Imidazole compounds

| Ref. | Structure | Chemical name |
|---|---|---|
| SN262 | 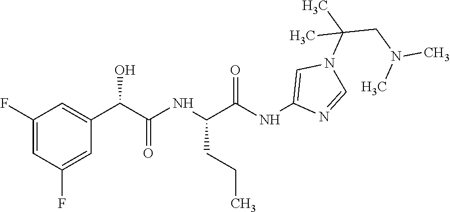 [SMILES: CCC[C@H](NC(=O)[C@@H](O)c1cc(F)cc(F)c1)C(=O)Nc1cn(C(C)(C)CN(C)C)cn1] | (S)-2-((S)-2-(3,5- difluorophenyl)-2- hydroxyacetamido)-N-(1-(1- (dimethylamino)-2- methylpropan-2-yl)-1H- imidazol-4-yl)pentanamide |
| SN326 | 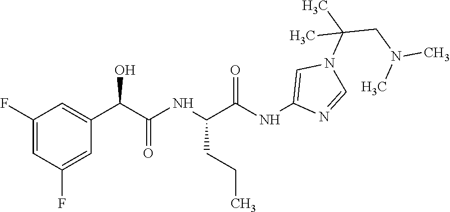 [SMILES: CCC[C@H](NC(=O)[C@H](O)c1cc(F)cc(F)c1)C(=O)Nc1cn(C(C)(C)CN(C)C)cn1] | (S)-2-((R)-2-(3,5- difluorophenyl)-2- hydroxyacetamido)-N-(1-(1- (dimethylamino)-2- methylpropan-2-yl)-1H- imidazol-4-yl)pentanamide |
| SN408 | 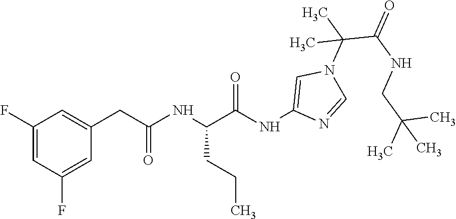 [SMILES: CCC[C@H](NC(=O)Cc1cc(F)cc(F)c1)C(=O)Nc1cn(C(C)(C)C(=O)NCC(C)(C)C)cn1] | (S)-2-(2-(3,5-difluorophenyl)- acetamido)-N-(1-(2-(methyl-1- (neopentylamino)-1- oxopropan-2-yl)-1H-imidazol-4- yl)pentanamide |

Screening and Testing of Imidazole Compounds

Western blot was applied for Val1744 (S3) on 293 TREX cells with FL-mNotch1-L1594P-6MT after treatment with imidazole compounds SN262, SN326 or SN408.

Subsequently, a dual luciferase assay of ADAM10/17dKO mEF cells, devoid of physiological ligand dependent signalling, transduced with mNotch1-LN-L1594P-6MT a Notch transcriptional reporter driving firefly luciferase and Green Fluorescent Protein (12xCSL-GFP-Luc) and TK-renilla luciferase construct. The ratio between Fluc and Rluc determines the activity of the imidazole compound tested, under indicated conditions.

Presenilin Specific Assays: Reconstituted DKO mEF

Immunoblot on cell lysates of PS1/2 DKO cells, and PS1/2 DKO reconstituted with either Presenilin 1 or Presenilin 2 were provided. Blots were probed with antibodies for nicastrin, Presenilin 1 (C-terminal fragment), Presenilin 2 (C-terminal fragment), and β-actin (loading control) to verify the cell construct used for later experiments. Double knock out of PSEN1 and PSEN2 and reconstitution were successful.

Presenilin Specific Assays: Luciferase/Cleavage Reporter Assay

Cells were plated 24 hours prior to transfection in 6 wells plates (200.000 cells/well in 2 mL). Cells were transfected using linear polyethylenimin (P-PEI) and DMEM/f12. 2.3 μg Firefly luciferase reporter plasmids containing synthetic Gal4-binding sites (5xUAS FR-luc) were used per 6 well, as well as 150 ng herpes thymidine kinase-driven Renilla luciferase (here serving as a housekeeping gene, and thus normalizing yardstick) and 50 ng Histone-2B (H2B)-GFP (here serving as a control for transfection success). In addition, 500 ng of plasmid DNA containing truncated (lacking the extracellular EGF repeats), constitutively active NOTCH1 receptor bearing the L1594P mutation (present in T-ALL), fused with Gal4 (binding to Firefly luciferase promoter sequence) and VP16 (transactivation co-factor recruiter) was added per 6 well. 6 hours after P-PEI transfection, cells were washed with PBS, resuspended in DMEM/f12+10% FCS+0.1% P/S with 0.1% treatment: 18 hours with 200 nM Dibenzazepine (DBZ) (Syncom, The Netherlands), DMSO (Sigma-Aldrich), and novel gamma-secretase inhibitors at given concentrations and replated in 12 wells plates to produce technical replicates (±50.000 cells/well in 1 mL). 24 h after transfection, 12 wells plates were washed with PBS, lysed and measured following manufacturer's directions for the Dual Luciferase Reporter (DLRTM) Assay system (Promega, The Netherlands). Analysis was carried out using the FLUOstar microplate reader (BMG Labtech, Germany). All values are given as fold Firefly/Renilla (RLU). All error bars represent mean±standard deviations of three independent experiments, each comprising three technical replicates, and are corrected for DMSO values.

### DESCRIPTION OF THE FIGURES

FIG. 1 γ-secretase complex composition regulates wild type Notch1 receptor cleavage and activation

FIG. 1A: Western blot analysis of Notch1 in QKO cells and QKO cells reconstituted with different γ-secretase subunits, Psen1:Aph1A (1A), Psen1:Aph1B (1B), Psen2:AphA (2A) and Psen2:Aph1B (2B) after stimulation with Dll4-Fc in the presence or absence of DBZ. The Notch1 C-20 antibody is directed against the c-terminal 20 amino acids of Notch1, detecting all cleaved forms of the receptor, whereas the valine1744 NICD1 antibody only recognizes Notch1 fragments cleaved at Valine1744 (NICD1/S3);

FIG. 1B: Western blot analysis of valine 1744 cleaved Notch1 in QKO, 1A, 1B, 2A and 2B cells after stimulation with DI11-Fc in the presence or absence of DBZ;

FIG. 1C: Different concentrations of NOTCH1-Gal4 were introduced into QKO, 1A, 1B, 2A and 2B MEFs and Gal4 reporter assay was performed after Dll4 stimulation;

FIG. 1D: NOTCH transcriptional activation measured by RBP-jK/CSL-luciferase reporter gene activation in QKO, 1A, 1B, 2A and 2B cells stimulated by Dll4 in the presence or absence of DBZ;

FIG. 1E: Notch transcriptional activation overtime measured by RBP-jκ/CSL-Gaussia reporter gene activation in QKO, 1A, 1B, 2A and 2B cells stimulated by Dll4; and

FIG. 1F: Hes1 and Hey1 mRNA expression levels in QKO, 1A, 1B, 2A and 2B cells after stimulation with Dll4 corrected with 18S. Lamin A/C and actin are used as loading controls. RLU; relative light units. Data are presented as mean±SEM for n=3.

FIG. 2 Psen2:Aph1B efficiently cleaves oncogenic mutant Notch1 receptor

FIG. 2A: Western blot analysis of ΔEGF-Notch1-L1594P-MYC in QKO cells and QKO cells reconstituted with different γ-secretase subunits, Psen1:Aph1A (1A), Psen1:Aph1B (1B), Psen2:Aph1A (2A) and Psen2:Aph1B (2B) in the presence or absence of DBZ. Immunoblotting against the C-terminal MYC-tag detects all cleaved forms of the receptor, whereas the valine1744 NICD1 antibody only recognizes Notch1 cleaved fragments cleaved at valine1744 (NICD1/S3);

FIG. 2B: Different concentrations of NOTCH1-L1594P-Gal4 were introduced into QKO, 1A, 1B, 2A and 2B MEFs and Gal4 reporter assay was performed;

FIG. 2C: NOTCH transcriptional activation measured by RBP-jκ/CSL-luciferase reporter gene activation in QKO, 1A, 1B, 2A and 2B cells expressing ΔEGF-Notch1-L1594P-MYC in the presence or absence of DBZ;

FIG. 2D: Notch transcriptional activation overtime measured by RBP-jκ/CSL-Gaussia reporter gene activation in QKO, 1A, 1B, 2A and 2B cells; and

FIG. 2E: Hes1 and Hey1 mRNA expression levels in QKO, 1A, 1B, 2A and 2B cells corrected with 18S. Lamin A/C is used as loading control. RLU; relative light units. Data are represented as mean±SEM for n=3.

FIG. 3 Differential 12XCSL activity inhibition in different Psen:Aph1 reconstituted cells upon CQ treatment

FIG. 3A: Inhibitory effect by CQ treatment of NOTCH transcriptional activity measured by RBP-jκ/CSL-luciferase reporter gene activation in QKO, 1A, 1B, 2A and 2B cells stimulated by Dll4; and

FIG. 3B: Inhibitory effect by CQ treatment of NOTCH transcriptional activity measured by RBP-jκ/CSL-luciferase reporter gene activation in QKO, 1A, 1B, 2A and 2B cells expressing ΔEGF-Notch1-L1594P-MYC.

FIG. 4 GSI Presenilin specificity

FIG. 4 shows the GSI Presenilin-2 specificity for compound SN262. Specific Psen2 activity, i.e. high percentage of cleavage inhibition, is shown compared to the percentage of cleavage inhibition of the same compound for Psen1.

FIG. 5 Schematic drawing of the Notch signalling pathway.

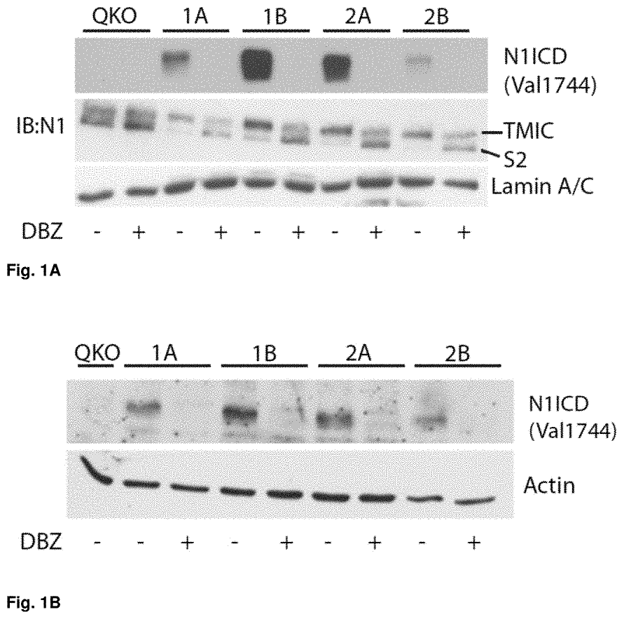

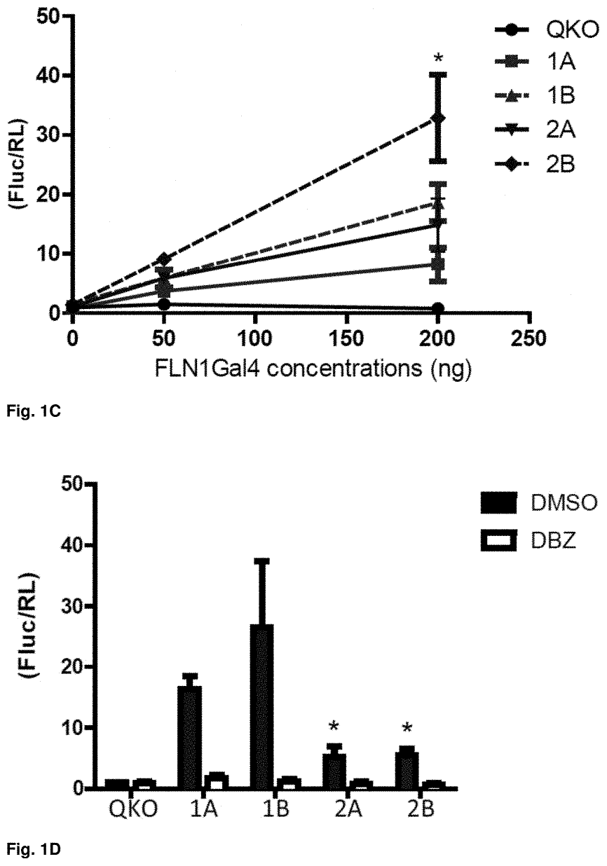

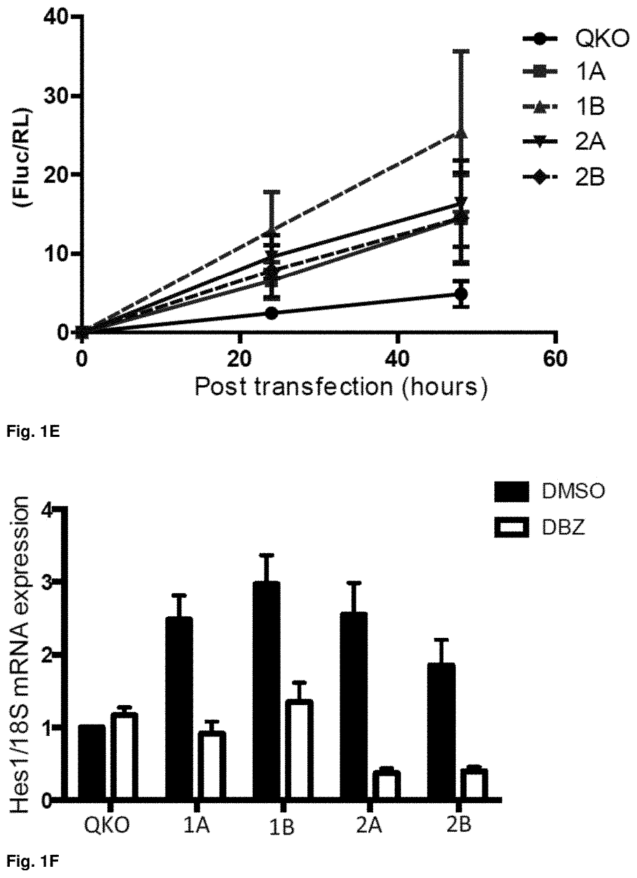

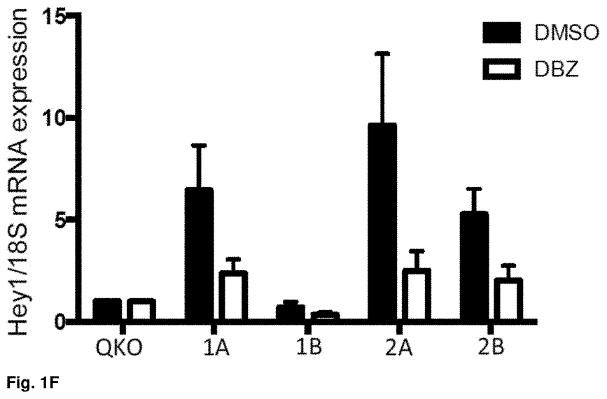

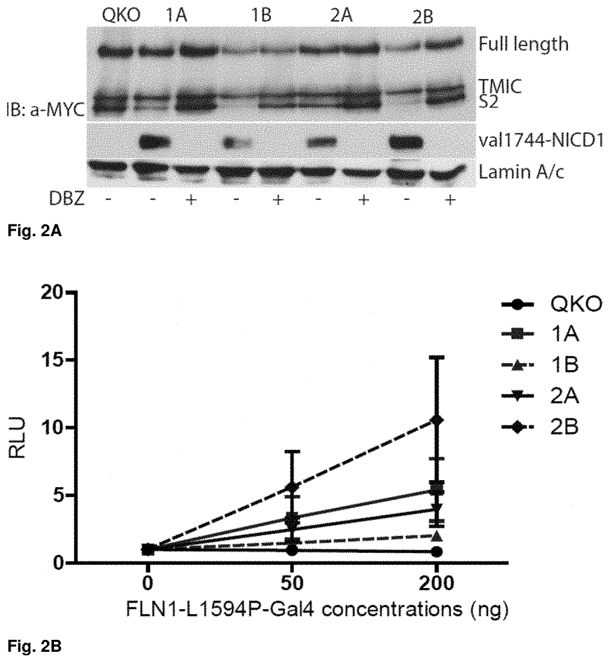

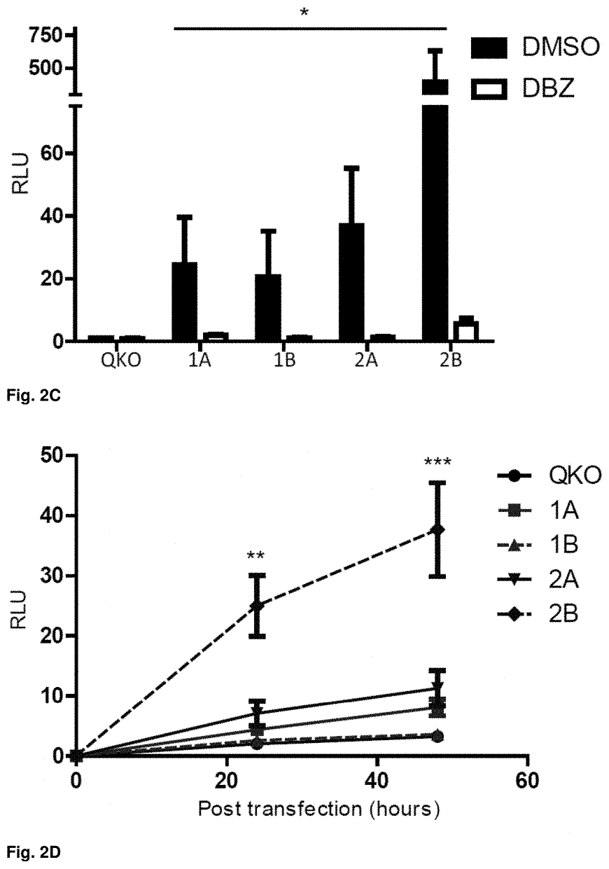

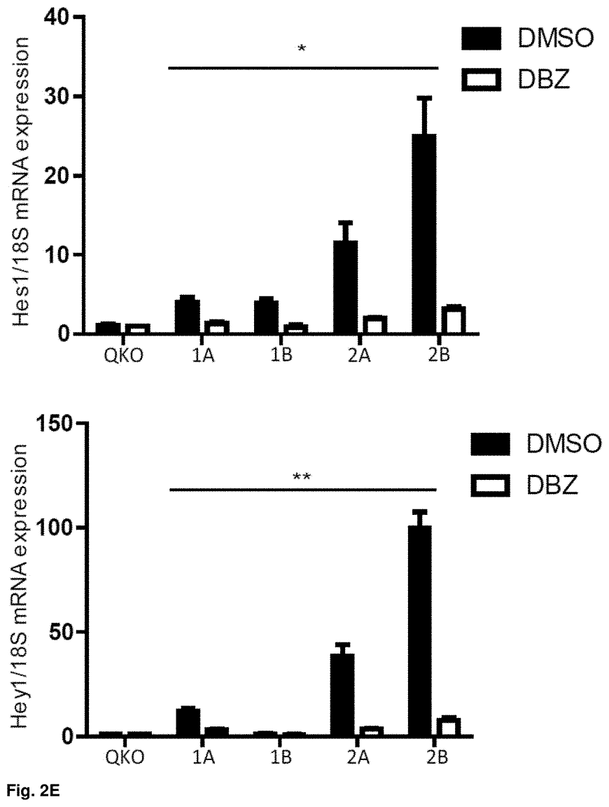

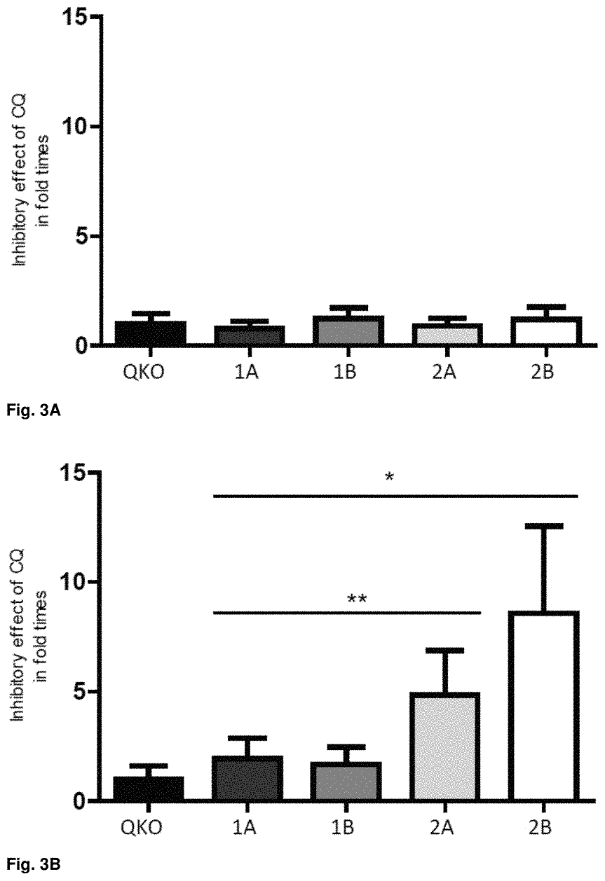

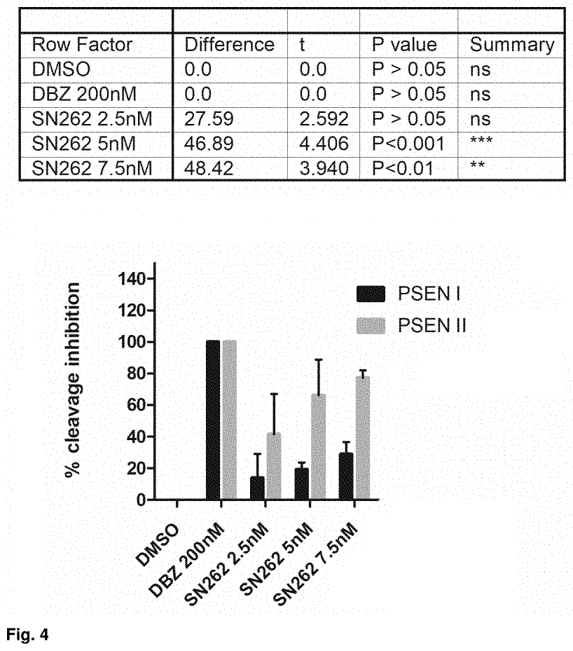

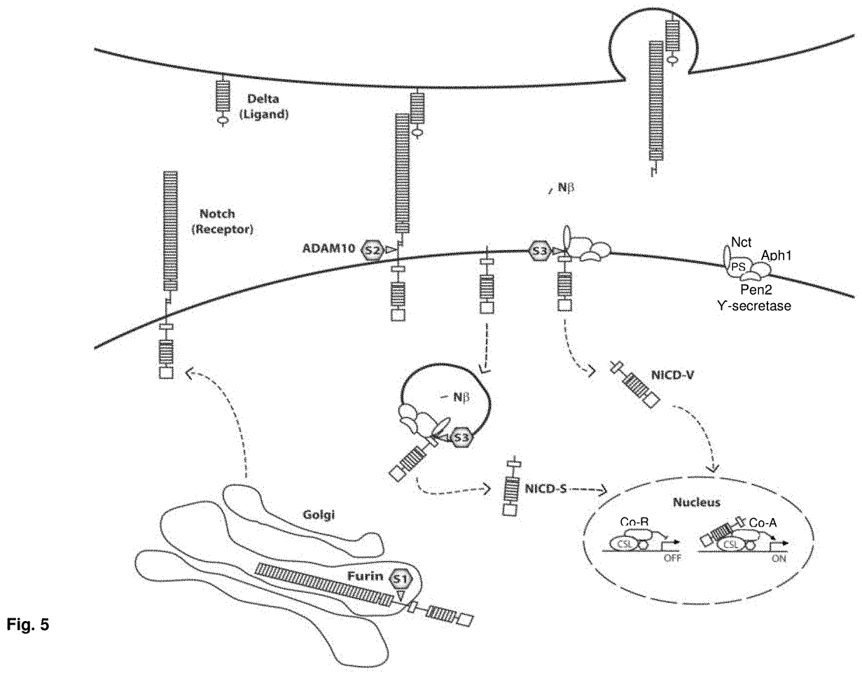

In the schematic drawing of FIG. 5, the Notch signalling pathway showing the proteolytic events. During maturation and transport of the receptor to the membrane Notch gets cleaved in the Golgi system at Site-1 (S1) by a furin-like convertase, resulting in a heterodimeric transmembrane receptor. At the cell surface in the absence of ligand the receptor is proteolysis-resistant. Only upon binding of ligand, inducing a substantial conformational change, the metalloprotease ADAM10 is able to cleave Notch at the newly exposed Site-2 (S2). This sheds off the NECD which can be transendocytosed into the ligand-expressing cell. On the receptor-expressing cell S2 cleavage results in a NEXT fragment that either is directly cleaved at the membrane at Val1744 (NICD-V) by the γ-secretase complex, or NEXT is internalized and processed in endocytic compartments by γ-secretase at Ser1747 (NICD-S). NICD translocates to the nucleus where it participates in a co-activator complex binding to CSL and starts target gene transcription. An additional cleavage at Site-4 (S4) is mediated by the γ-secretase in the centre of the transmembrane domain resulting in the N-terminal fragment, possibly needed to clear residual Notch fragments from the plasma membrane.

1. Selective presenilin-2 γ-secretase inhibitor for use in the treatment of diseases associated with a defect leading to Notch receptor hyperactivity.

2. Selective presenilin-2 γ-secretase inhibitor for use according to claim 1, wherein the diseases associated with a defect leading to Notch receptor hyperactivity comprise diseases associated with an aberrant Notch signalling pathway including disease associated with:
gain-of-function mutations of the Notch receptor;
loss-of-function or gain-of-function mutations affecting the activity of the Notch receptor;
chromosomal rearrangements in the Notch signalling pathway; or
defects in protein trafficking altering the subcellular localisation of the Notch receptor molecules.

3. Selective presenilin-2 γ-secretase inhibitor for use according to claim 1, wherein the diseases associated with a defect leading to Notch receptor hyperactivity comprise diseases associated with oncogenic ligand-independent gain-of-function mutations of the Notch receptor or loss-of-function or gain-of-function mutations leading to hyperactivity of the Notch receptor.

4. Selective presenilin-2 γ-secretase inhibitor for use according to claim 1, wherein the diseases associated with a defect leading to Notch receptor hyperactivity comprise diseases associated with a defect leading to Notch1 receptor hyperactivity, such as oncogenic ligand-independent gain-of-function mutations of the Notch 1 receptor or loss-of-function or gain-of-function mutations leading to hyperactivity of the Notch receptor.

5. Selective presenilin-2 γ-secretase inhibitor for use according to claim 1, wherein the diseases associated with a defect leading to Notch receptor hyperactivity are cancers selected from the group consisting of leukemia, breast cancer, prostate cancer, colorectal cancer, penile cancer, Head and Neck Cancer, lung cancer, oesophageal cancer, liver cancer, ovarian, cervical and endometrial cancer, brain cancer, bladder cancer, skin cancer, bone cancer, disseminated diseases, such as metastatic cancer, and the like.

6. Selective presenilin-2 γ-secretase inhibitor for use according to claim 1, wherein the diseases associated with a defect leading to Notch receptor hyperactivity are haematological malignancies selected from the group consisting of T-cell acute lymphoblastic leukaemia (T-ALL), and B-cell chronic lymphocytic leukaemia.

7. Selective presenilin-2 γ-secretase inhibitor for use according to claim 1, wherein the diseases associated with a defect leading to Notch receptor hyperactivity are respiratory diseases selected from the group consisting of asthma, pulmonary fibrosis, and non-small cell lung carcinoma.

8. Selective presenilin-2 γ-secretase inhibitor for use according to claim 1, wherein the selective presenilin-2 γ-secretase inhibitor is selective in inhibiting the presenilin-2, anterior pharynx-defective 1 γ-secretase complex.

9. Selective presenilin-2 γ-secretase inhibitor for use according to claim 1, wherein the selective presenilin-2 γ-secretase inhibitor is identified by a screening method comprising the steps of:
providing a candidate for selectively inhibiting presenilin-2 γ-secretase complex;
contacting the candidate with a presenilin-1 γ-secretase complex and presenilin-2 γ-secretase complex; and
identifying the candidate selectively inhibiting presenilin-2 γ-secretase as the selective presenilin-2 γ-secretase inhibitor

10. 4-aminoquinolines as selective presenilin-2 γ-secretase inhibitors for use in the treatment of a disease associated with a defect leading to Notch receptor hyperactivity, such as oncogenic ligand-independent gain-of-function mutations of the Notch1 receptor or loss-of-function or gain-of-function mutations leading to hyperactivity of the Notch receptor, wherein the 4-aminoquinolines are selected from compounds of formula: 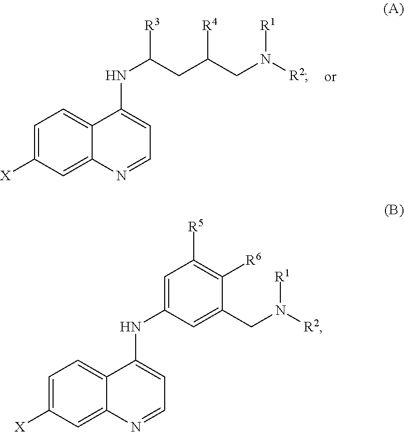
wherein:
X is selected from hydrogen, fluoro, chloro, bromo, iodo, hydroxy, nitro, cyano, amino, C₁₋₄ alkyl, C₁₋₄ alkoxy, C₁₋₄ alkylamino, C₁₋₄ alkylthio, aryl, benzyl, phenoxy, or benzoxy;
R¹ and R² are independently selected from hydrogen, C₁₋₄ alkyl, aryl, benzyl, C₁₋₄ alkylol, or alkylphenol;
R³ and R⁴ are independently selected from hydrogen, C₁₋₄ alkyl, aryl, benzyl, or C₁₋₄ alkylol; and
R⁵ and R⁶ are independently selected from hydrogen, fluoro, chloro, bromo, iodo, hydroxy, nitro, cyano, amino, or C₁₋₄ alkyl.

11. 4-aminoquinolines for use according to claim 10, wherein:
X is selected from chloro;
R¹ and R² are independently selected from ethyl or 2-hydroxyethyl;
R³ and R⁴ are independently selected from hydrogen or methyl; and
R⁵ and R⁶ are independently selected from hydrogen or hydroxyl.

12. 4-aminoquinolines for use according to claim 10 selected from the group consisting of amodiaquine, chloroquine, and hydroxychloroquine.

13. Imidazole compounds as selective presenilin-2 γ-secretase inhibitors, wherein the imidazole compounds are selected from compounds of formula: 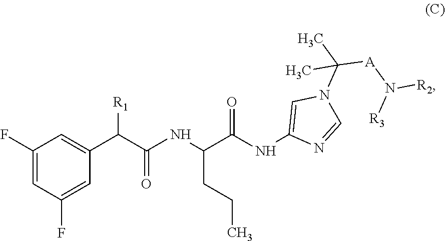
wherein:
R₁ is selected from hydrogen, or hydroxy;
A is selected from —CH₂— or —CO—; and
R₂ and R₃ are independently selected from hydrogen, methyl, ethyl, propyl, isopropyl, tert-butyl, or neopentyl,
with the proviso that when R₁ is hydrogen, A is —CO—.

14. Imidazole compound according to claim 13, wherein:
R₁ and R₂ are selected from hydrogen, A is selected from —CO— and R₃ is selected from neopentyl; or
R₁ is selected from hydroxyl, A is selected from —CH₂—, and R₂ and R₃ are selected from methyl.

15. Imidazole compound according to claim 13 selected from the group consisting of:
(S)-2-((S)-2-(3,5-difluorophenyl)-2-hydroxyacetamido)-N-(1-(1-(dimethylamino)-2-methylpropan-2-yl)-1H-imidazol-4-yl)pentanamide,
(S)-2-((R)-2-(3,5-difluorophenyl)-2-hydroxyacetamido)-N-(1-(1-(dimethylamino)-2-methylpropan-2-yl)-1H-imidazol-4-yl)pentanamide, and
(S)-2-(2-(3,5-difluorophenyl)-acetamido)-N-(1-(2-(methyl-1-(neopentylamino)-1-oxopropan-2-yl)-1H-imidazol-4-yl)pentanamide.

16. Imidazole compound according to claim 13 for use as a medicament.

17. Imidazole compound according to claim 13 for use in the treatment of a disease associated with a defect leading to Notch receptor hyperactivity.

18. Pharmaceutical composition comprising the imidazole compound according to claim 13.
